(19) United States
(12) Patent Application Publication
(10) Pub. No.: US 2024/0336919 A1
(43) Pub. Date: Oct. 10, 2024

(54) RNA EDITING

(71) Applicant: Hoffmann-La Roche Inc., Little Falls, NJ (US)

(72) Inventors: Lars JOENSON, Viby Sjaelland (DK); Tobias MERKLE, Copenhagen (DK); Anja MOELHART HOEG, Hillerød (DK); Jonas VIKESAA, Fredensborg (DK)

(73) Assignee: Hoffmann-La Roche Inc., Little Falls, NJ (US)

(21) Appl. No.: 18/618,604

(22) Filed: Mar. 27, 2024

Related U.S. Application Data
(63) Continuation of application No. PCT/EP2022/076748, filed on Sep. 27, 2022.

(30) Foreign Application Priority Data
Sep. 29, 2021 (EP) 21199761.4

(51) Int. Cl. C12N 15/113

(52) U.S. Cl. CPC C12N 15/113; C12N 2310/11; C12N 2310/315; C12N 2310/321; C12N 2320/30

(57) ABSTRACT

The present invention relates to an antisense oligonucleotide for use in treating and/or preventing a polyglutamine (polyQ) disease, wherein said antisense oligonucleotide is capable of specifically binding to a CAG repeat region of an mRNA encoding a polyQ disease-related protein such that a double stranded RNA is formed which is capable of attracting an Adenosine Deaminase Acting on RNA (ADAR) inserting an A to I exchange into at least one CAG trinucleotide of the CAG repeat region of said mRNA. The present invention further relates to a pharmaceutical composition comprising said antisense molecule.

### CROSS REFERENCE TO RELATED APPLICATIONS

This application is a continuation of International Application No. PCT/EP2022/076748, having an international filing date of Sep. 27, 2022, which claims benefit to European Patent Application No. EP21199761.4, filed Sep. 29, 2021, the contents of which are each incorporated herein by reference in their entirety.

### SEQUENCE LISTING

This application contains a Sequence Listing which has been submitted electronically in XML format and is hereby incorporated by reference in its entirety. Said XML copy, created on Mar. 20, 2024, is named P36581-US_Seq_List.xml and is 37,186 bytes in size.

### FIELD OF INVENTION

The present invention relates to an antisense oligonucleotide for use in treating and/or preventing a polyglutamine (polyQ) disease, wherein said antisense oligonucleotide is capable of specifically binding to a CAG repeat region of an mRNA encoding a polyQ disease-related protein such that a double-stranded RNA is formed which is capable of attracting an Adenosine Deaminase Acting on RNA (ADAR) inserting an A to I exchange into at least one CAG trinucleotide of the CAG repeat region of said mRNA. The present invention further relates to a pharmaceutical composition comprising said antisense molecule.

### BACKGROUND

Polyglutamine (PolyQ) diseases are driven by an elongation of a CAG repeat region within a certain gene for each disease. This results in the formation of long stretches of glutamine in the protein. Polyglutamine (Poly Q) repeats are known to make proteins prone to formation of aggregates and in some cases fibrillary aggregates, that cause disease.

The polyglutamine family of diseases encompasses several diseases such as Huntington's disease (HD), Spinocerebellar ataxia (SCA) type 1, type 2, type 3 (also known as Machado-Joseph disease), type 6, type 7, and type 17, Dentatorubralpallidoluysian atrophy and Kennedy's disease.

Spinocerebellar ataxia-type 3 (SCA3) is an autosomal dominant, progressive neurodegenerative disorder with variable age of onset and severity. SCA3 was originally described in people of Portuguese descent, and in particular from the Azores islands where SCA3 is most prevalent (e.g., the incidence of SCA3 is 1/140 in the small island of Flores) (Sudarsky L., et al., Clin. Neurosci. 1995; 3:1 7-22). SCA3 was subsequently identified in several other countries and is now considered to be the most common dominantly inherited hereditary ataxia. Clinically, patients with SCA3 present with progressive gait and limb ataxia, dysarthria and a variable combination of other symptoms including pyramidal signs, dystonia, oculomotor disorders, faciolingual weakness, neuropathy, progressive sensory loss and parkinsonian features. In its more severe forms, SCA3 is characterized by defects in both pyramidal (e.g., motor, somatosensory) and extrapyramidal (e.g., muscle tone) neural functions. Within affected families, this form of ataxia also demonstrates an anticipation effect, which is characterized by an earlier disease onset and more severe symptoms with each new affected generation.

All forms of SCA3 are attributable to an unstable and iterative genetic expansion of a (CAG) n tract in the coding region of ATXN3 on chromosome 14q32.1 that encodes a pathogenic polyglutamine region or tract in the translated ATXN3 protein (Kawaguchi Y., et al., Nature Genet. 1994; 8:221-228). The unstable and iterative expansion of the (CAG) n tract in the coding region of ATXN3 (and the pathogenic poly-glutamine tract encoded thereby) causes an increase in protein misfolding, which results in aggregation and formation of nuclear and cytoplasmic inclusions (Paulson et al., 1997, Neuron 19, 333-344).

Specifically, the expansion of these repeats from the normal 13-36 to 68-79 is the cause of Machado-Joseph disease. There is an inverse correlation between the age of onset and CAG repeat numbers. The CAG repeats are translated into a poly Q (polyglutamine) stretch in the ATXN3 protein, which if long enough can cause the protein to aggregate. Currently, there is no cure for SCA3. However, some symptoms can be treated. For example, spasticity can be treated with baclofen.

Huntington's disease (HD) is a polyglutamine disease as well. It is a neurodegenerative disease that is typically inherited from an affected parent who carries a mutation in the huntingtin gene (HTT). As for SCA3, there is currently no cure for Huntington's disease. However, some symptoms, such as movement problems, can be treated.

Moore et al. reported that antisense oligonucleotides (ASOs) targeting ATXN3 were capable of reducing levels of the pathogenic ATXN3 protein both in human disease fibroblasts and in a mouse model expressing the full-length human mutant ATXN3 gene (Moore et al., Mol Ther Nucleic Acids. 2017; 7:200-210).

Toonen et al. used antisense oligonucleotides to mask predicted exonic splicing signals of ATXN3, resulting in exon 10 skipping from ATXN3 pre-mRNA. The skipping of exon 10 led to formation of a truncated ataxin-3 protein lacking the toxic polyglutamine expansion, but retaining its ubiquitin binding and cleavage function.

WO2013/138353, WO2018/089805 disclose antisense oligonucleotides targeting human ATXN3 mRNA for use in the treatment of SCA3.

WO2015/017675 discloses a compound comprising a single-stranded oligonucleotide consisting of 13 to 30 linked nucleosides and having a nucleobase sequence complementary to a repeat region of an expanded repeat-containing target RNA, wherein the 5 ‘-terminal nucleoside of the single-stranded oligonucleotide comprises a phosphate moiety and an internucleoside linking group linking the 5’-terminal nucleoside to the remainder of the oligonucleotide.

Gagnon et al. showed that antisense oligonucleotides targeting the expanded CAG repeat resulted in allele-selective inhibition of mutant huntingtin expression. Antisense oligonucleotides incorporating a variety of modifications, including bridged nucleic acids and phosphorothioate internucleotide linkages, exhibited allele-selective silencing in patient-derived fibroblasts (Gagnon et al., 2010 Nov. 30;49 (47): 10166-78).

Yu et al. showed that chemically modified single-stranded siRNAs (ss-siRNAs) targeting the CAG-repeat are potent and allele-selective inhibitors of mutant huntingtin (HTT) expression in cells derived from Huntington disease patients. Placement of mismatched bases mimicked micro-RNA recognition and optimized discrimination between mutant and wild-type alleles (Yu et al., Cell. 2012 Aug. 31;150 (5): 895-908).

Adenosine deaminases acting on RNA (ADARs) are editing enzymes that convert adenosine to inosine in duplex RNA (Matthews et al. Nat Struct Mol Biol. 2016 May;23 (5): 426-33). Thus, they are capable of introducing post-transcriptional modifications into an mRNA transcript. Inosine is interpreted by the cell to be guanosine. Human ADAR has 2-3 amino-terminal dsRNA binding domains (dsRBDs) and one carboxy-terminal catalytic deaminase domain. Engineered antisense oligonucleotides could be used for site-directed RNA editing.

WO 2016/097212 A1 discloses an oligonucleotide construct for the site-directed editing of a nucleotide in a target RNA sequence in a eukaryotic cell, said oligonucleotide construct comprising: (a) a targeting portion, comprising an antisense sequence complementary to part of the target RNA; and (b) a recruiting portion capable of binding and recruiting an RNA editing entity naturally present in said cell and capable of performing the editing of said nucleotide.

WO 2017/220751 A1 discloses an antisense oligonucleotide capable of forming a double-stranded complex with a target RNA in a cell, for the deamination of a target adenosine present in the target RNA by an ADAR enzyme present in the cell, wherein:
a) the antisense oligonucleotide is complementary to a target RNA region comprising the target adenosine, and the antisense oligonucleotide AON optionally comprises one or more mismatches, wobbles and/or bulges with the complementary target RNA region;
b) the antisense oligonucleotide comprises one or more nucleotides with one or more sugar modifications, provided that the nucleotide opposite the target adenosine comprises a ribose with a 2′-OH group, or a deoxyribose with a 2′-H group;
c) the antisense oligonucleotide does not comprise a portion that is capable of forming an intramolecular stem-loop structure that is capable of binding an ADAR enzyme;
d) the antisense oligonucleotide does not include a 5′-terminal 06-benzylguanine modification;
e) the antisense oligonucleotide does not include a 5′-terminal amino modification; and
f) the antisense oligonucleotide is not covalently linked to a SNAP-tag domain.

WO 2018/134301 A1 discloses a double stranded oligonucleotide complex comprising an antisense oligonucleotide (AON) and a complementary sense oligonucleotide (SON) annealed to the AON via Watson-Crick base-pairing, for use in ADAR-mediated targeted deamination of a target adenosine in a target RNA sequence in a cell by an ADAR enzyme present in the cell.

WO 2018/041973 A1 discloses an antisense oligonucleotide capable of forming a double stranded complex with a target RNA sequence in a cell for the deamination of a target adenosine in the target RNA sequence by an ADAR enzyme present in the cell, wherein (i) the antisense oligonucleotide capable comprises a Central Triplet of 3 sequential nucleotides, (ii) the nucleotide directly opposite the target adenosine is the middle nucleotide of the Central Triplet, (iii) the middle nucleotide of the Central Triplet is a cytidine, and (iv) 1, 2 or 3 nucleotides in the Central Triplet comprise a sugar modification and/or a base modification.

WO 2019/158475 A1 discloses an editing oligonucleotide (EON) capable of forming a double stranded complex with a target RNA molecule in a cell, and capable of recruiting an endogenous enzyme with ADAR activity, wherein:
the target RNA molecule comprises a target adenosine for deamination by the enzyme with ADAR activity;
the EON comprises a Central Triplet of three sequential nucleotides in which the nucleotide directly opposite the target adenosine is the middle nucleotide (position 0) of the Central Triplet and wherein the positions are positively (+) and negatively (−) incremented towards the 5′ and 3′ ends of the EON, respectively;
the EON comprises a nucleotide at position 0 that mismatches with the target adenosine;
the EON comprises one or more nucleotides comprising a 2′-O-methoxyethyl (2′-MOE) ribose modification.

To our knowledge, antisense oligonucleotides (ASOs) that recruit the endogenous RNA editing enzyme ADAR (adenosine deaminase acting on RNA) to perform A-to-I (adenosine to inosine) in CAG repeats in the mRNA encoding polyQ disease-related proteins have not yet been described.

### Objective of the Invention

The present invention describes antisense oligonucleotides (ASOs) that recruit the endogenous RNA editing enzyme ADAR (adenosine deaminase acting on RNA) to perform A-to-I (adenosine to inosine) exchanges in one or more adenosine bases of CAG repeats on mRNA level. Since inosine is read by the translational machinery as guanosine, one or more CAG trinucleotides are converted to CGG trinucleotides on the mRNA level. This results in multiple interruptions of the long disease causing polyQ tracts by arginine and may reduce the toxic aggregation of polyQ disease-related proteins.

The present invention also describes antisense oligonucleotides for treating and/or preventing a polyglutamine (polyQ) disease.

### SUMMARY OF INVENTION

The present invention relates to an antisense oligonucleotide, wherein said oligonucleotide is capable of specifically binding to a CAG repeat region of an mRNA encoding a polyQ disease-related protein such that a double-stranded RNA is formed which is capable of attracting an Adenosine Deaminase Acting on RNA (ADAR) inserting an A to I exchange into at least one CAG trinucleotide of the CAG repeat region of said mRNA.

In an embodiment, said antisense oligonucleotide is an RNA antisense oligonucleotide, such as a single-stranded RNA antisense oligonucleotide.

The present invention also relates to an antisense oligonucleotide as set forth above for treating and/or preventing a polyglutamine (polyQ) disease, such as Huntington's disease (HD), Spinocerebellar ataxia (SCA) 1, 2, 3, 6, 7, or 17, Dentatorubralpallidoluysian atrophy, and Kennedy's disease.

The present invention also relates to a polyQ disease-associated polypeptide comprising one or more arginine residues in its polyQ tract.

The present invention further pertains to a pharmaceutical composition comprising the antisense oligonucleotide of the present invention and a pharmaceutically acceptable excipient.

A further aspect of the present invention is an ex vivo or in vivo method for preventing aggregation of the polyQ disease-related protein comprising contacting cells expressing a polyQ disease-related protein with the antisense oligonucleotide as of the present invention.

In a further aspect, the invention provides methods for treating or preventing polyglutamine (polyQ) disease comprising administering a therapeutically or prophylactically effective amount of the antisense oligonucleotide of the present invention, such as the antisense oligonucleotide or siRNA of the invention to a subject suffering from or susceptible to the disease.

### BRIEF DESCRIPTION OF THE FIGURES

FIG. 1: Representative fraction of the reads in a PBS sample (A) and a sample treated with an antisense oligonucleotide (ASO) with CMP ID NO: 4 (see Table 1). (B). A dot “.” represents a match to the reference sequence, and a mismatch is shown by the new base. An A->G editing has occurred multiple times in the ASO treated cells.

### DEFINITIONS

### Polyglutamine (PolyQ) Disease

The term “polyQ disease” or “polyglutamine disease” as used herein refers to a trinucleotide repeat disorder in which the codon CAG is repeated in the coding region of a gene resulting in a length of polyQ tract beyond normal. PolyQ diseases known to date include those listed in the Table A below (based on Den Dunnen W. F. A. Handbook of Clinical Neurology Vol. 145, 2018). Table A indicates the type of polyQ disease, the gene encoding the polyQ disease-related protein, the normal length of the CAG tract of said gene (for example, a value of 50 means that the CAG tract comprises 50 CAG codons) and the pathogenic (i.e. disease-causing) length of the CAG tract of said gene.

TABLE A
PolyQ diseases

| Type | Gene | Normal | Pathogenic |
|---|---|---|---|
| Dentatorubropallidoluysian atrophy (DRPLA) | ATN1 or | 6-35 | 49-88 |
| | DRPLA | | |
| Huntington disease (HD) | HTT | 6-35 | 36-250 |
| Spinobulbar muscular atrophy (SBMA) | AR | 9-36 | 38-62 |
| Spinocerebellar ataxia type 1 (SCA1) | ATXN1 | 6-35 | 49-88 |
| Spinocerebellar ataxia type 2 (SCA2) | ATXN2 | 14-32 | 32->100 |
| Spinocerebellar ataxia type 3 or Machado-Joseph | ATXN3 | 12-40 | 55-86 |
| disease (SCA3) | | | |
| Spinocerebellar ataxia type 6 (SCA6) | CACNA1A | 4-18 | 20-33 |
| Spinocerebellar ataxia type 7 (SCA7) | ATXN7 | 7-17 | 37-306 |
| Spinocerebellar ataxia type 12 (SCA12) | PPP2R2B | 7-28 | 55-78 |
| Spinocerebellar ataxia type 17 (SCA17) | TBP | 25-42 | 47-63 |

In an embodiment of the present in invention, the polyQ disease is selected from the group consisting of: Huntington's disease (HD), Spinobulbar muscular atrophy (SMBA), Spinocerebellar ataxia (SCA) 1, 2, 3, 6, 7, 12 or 17, Dentatorubralpallidoluysian atrophy, and Kennedy's disease.

In some embodiments, the polyQ disease is Huntington's disease.

In some embodiments, the polyQ disease is SCMA.

In some embodiments, the polyQ disease is SCA1.

In some embodiments, the polyQ disease is SCA2.

In some embodiments, the polyQ disease is SCA3 (also referred to as Machado-Joseph disease).

In some embodiments, the polyQ disease is SCA6.

In some embodiments, the polyQ disease is SCA7.

In some embodiments, the polyQ disease is SCA12.

In some embodiments, the polyQ disease is SCA17.

In some embodiments, the polyQ disease is Dentatorubralpallidoluysian atrophy.

In some embodiments, the polyQ disease is Kennedy's disease.

polyQ Disease-Related Protein

As set forth, elsewhere herein, polyglutamine diseases occur if a mutation causes a polyglutamine tract in a specific gene to become too long, i.e. if the length of the polyglutamine tract is pathogenic. The proteins associated with PolyQ disease are well-known in art. They are encoded by the genes shown in Table A above.

For Dentatorubropallidoluysian atrophy, the polyQ disease-associated protein is ATN1 (Atrophin-1). The amino acid sequence can be assessed via Uniprot, see P54259.

For Huntington disease (HD), the polyQ disease-associated protein is HTT (Huntingtin). The amino acid sequence of the human protein can be assessed via Uniprot, see P428.

For Spinobulbar muscular atrophy (SBMA), the polyQ disease-associated protein is the androgen receptor (AR), also known as NR3C4. The amino acid sequence of the human protein can be assessed via Uniprot, see P10275.

For Spinocerebellar ataxia type 1 (SCA1), the polyQ disease-associated protein is ATXN1 (Ataxin-1) The amino acid sequence of the human protein can be assessed via Uniprot, see P54253.

For Spinocerebellar ataxia type 2 (SCA2), the polyQ disease-associated protein is ATXN2 (Ataxin-2). The amino acid sequence of the human protein can be assessed via Uniprot, see Q99700.

For Spinocerebellar ataxia type 3 or Machado-Joseph disease (SCA3), the polyQ disease-associated protein is ATXN3 (Ataxin-3). The amino acid sequence of the human protein can be assessed via Uniprot, see P54252.

For Spinocerebellar ataxia type 6 (SCA6), the polyQ disease-associated protein is Cav2.1, also called the P/Q voltage-dependent calcium channel or CACNA1A. The amino acid sequence of the human protein can be assessed via Uniprot, O00555.

For Spinocerebellar ataxia type 7 (SCA7), the polyQ disease-associated protein is ATXN7 (Ataxin-7). The amino acid sequence of the human protein can be assessed via Uniprot, see O15265.

For Spinocerebellar ataxia type 12 (SCA12), the polyQ disease-associated protein is Serine/threonine-protein phosphatase 2A 55 kDa regulatory subunit B beta isoform (PPP2R2B). The amino acid sequence of the human protein can be assessed via Uniprot, see Q00005.

For Spinocerebellar ataxia type 17 (SCA17), the polyQ disease-associated protein is TATA-binding protein (TBP). The amino acid sequence of the human protein can be assessed via Uniprot, see P20226.

The polyQ disease-related protein shall comprise a polyglutamine tract having a length which pathogenic, i.e. which exceeds the normal length. The pathogenic length for the individual diseases is shown in Table A above.

In an embodiment, the polyQ disease-related protein is HTT, and the mRNA encoding said protein comprises 36 to 250 consecutive CAG repeats. Thus, the polyglutamine tract comprises 36 to 250 glutamine residues.

In an embodiment, the polyQ disease-related protein is ATXN1, and the mRNA encoding said protein comprises 49 to 88 consecutive CAG repeats. Thus, the polyglutamine tract comprises 49 to 88 glutamine residues.

In an embodiment, the polyQ disease-related protein is ATXN2, and the mRNA encoding said protein comprises 32 to 100 consecutive CAG repeats. Thus, the polyglutamine tract comprises 32 to 100 glutamine residues.

In an embodiment, the polyQ disease-related protein is ATXN3, and the mRNA encoding said protein comprises 55 to 86 consecutive CAG repeats. Thus, the polyglutamine tract comprises 55 to 86 glutamine residues.

In an embodiment, the polyQ disease-related protein is CACNA1A, and the mRNA encoding said protein comprises 49 to 88 consecutive CAG repeats. Thus, the polyglutamine tract comprises 49 to 88 glutamine residues.

In an embodiment, the polyQ disease-related protein is ATXN7, and the mRNA encoding said protein comprises 37 to 306 consecutive CAG repeats. Thus, the polyglutamine tract comprises 37 to 306 glutamine residues.

In an embodiment, the polyQ disease-related protein is PPP2R2B, and the mRNA encoding said protein comprises 55 to 78 consecutive CAG repeats. Thus, the polyglutamine tract comprises 55 to 78 glutamine residues.

In an embodiment, the polyQ disease-related protein is TBP, and the mRNA encoding said protein comprises 47 to 63 consecutive CAG repeats. Thus, the polyglutamine tract comprises 47 to 63 glutamine residues.

In an embodiment, the polyQ disease-related protein is ATN1 or DRPLA; and the mRNA encoding said protein comprises 49 to 88 consecutive CAG repeats. Thus, the polyglutamine tract comprises 49 to 88 glutamine residues.

In an embodiment, the polyQ disease-related protein is AR, and the mRNA encoding said protein comprises 38 to 62 consecutive CAG repeats. Thus, the polyglutamine tract comprises 38 to 62 glutamine residues.

### Adenosine Deaminase Acting on RNA (ADAR)

As used herein, the term “ADAR” or “Adenosine Deaminase Acting on RNA” refers to a double-stranded RNA specific adenosine deaminase (EC 3.5.4.37 which catalyzes the hydrolytic deamination of adenosine to inosine in double-stranded RNA (dsRNA), referred to as A to I editing. The catalyzed reaction is a follows:

Adenine in double-stranded RNA+H₂O=hypoxanthine in double-stranded RNA+NH₃

Hypoxanthine is the nucleobase in inosine.

Preferably, the ADAR polypeptide is the human ADAR polypeptide. Information on the human amino acid sequence can be found under UniProt reference number P55265. It is to be understood that the ADAR polypeptide shall be catalytically active. For example, the human ADAR1 and ADAR2 isoforms are known to be catalytically active.

In an embodiment, the ADAR polypeptide is the endogenous ADAR polypeptide. In another embodiment, the ADAR polypeptide is recombinantly expressed in the target cell. In yet another embodiment, the ADAR polypeptide has been delivered to the target cell.

The Adenosine Deaminase Acting on RNA (ADAR) inserting an A to I exchange into at least one CAG trinucleotide of the CAG repeat region of mRNA encoding a polyQ disease-related protein. The translation of the thus generated mRNA results in a polyQ disease-related protein in which the polyQ tract is interrupted (and thus comprises) by at least one arginine residue. In some embodiments, the polyQ tract of the generated protein is interrupted (and thus comprises) by 2, 3, 4, 5, 6 or more arginine residues. Thus, the generated polypeptide comprises in its polyQ tract one or more arginine residues (as described elsewhere herein).

### Target Nucleic Acid

According to the present invention, the target nucleic acid is an mRNA encoding a polyQ disease-related protein as set forth elsewhere herein. The term polyQ disease-related protein has been defined elsewhere herein. It is to be understood that the target nucleic acid is the region encoding the polyglutamine tract of the of the polyQ disease-related protein. Preferably, the polyglutamine tract has a disease causing length, i.e. a pathogenic length (see e.g. Table A). Thus, the target RNA shall comprise an expanded CAG repeat region.

### Target Sequence

The term “target sequence” as used herein refers to a sequence of nucleotides present in the target nucleic acid, which comprises the nucleobase sequence which is complementary to the oligonucleotide or nucleic acid molecule of the invention. In other words, the “target sequence” is the region in the target mRNA to which the antisense molecule of the present invention hybridizes.

In some embodiments, the target sequence consists of a region on the target nucleic acid with a nucleobase sequence that is complementary to the contiguous nucleotide sequence of the oligonucleotide of the invention. This region of the target nucleic acid may interchangeably be referred to as the target nucleotide sequence, target sequence or target region. In some embodiments, the target sequence is longer than the complementary sequence of a nucleic acid molecule of the invention.

The target sequence in accordance with the present invention is the CAG repeat region of an mRNA encoding a polyQ disease-related protein. Thus, the antisense oligonucleotide or contiguous nucleotide sequence as referred to herein shall be complementary to the CAG-repeat region. The number of CAG repeats comprised by the CAG repeat region may depend on the gene to be targeted. Specifically, the number shall be a pathogenic number as shown in Table A above. For ATXN3, the target sequence shall comprise at least 55 CAG repeats, such as 55 to 86 consecutive CAG repeats. Thus, the mRNA encoding the ATXN3 polypeptide shall comprise at least 55 CAG repeats, in particular at least about 55 consecutive CAG repeats. It is to be understood that the oligonucleotide of the present invention may not be complementary to all CAG repeats present in the CAG repeat region. However, it shall be complementary to a portion of the CAG repeat which is long enough in order for allowing ADAR to act.

In an embodiment, the oligonucleotide or contiguous nucleotide sequence is complementary, such as is at least 90% complementary, such as at least 95% complementary to a CAG repeat region of an mRNA encoding a polyQ disease-related protein.

For example, the antisense oligonucleotide, or contiguous nucleotide sequence, may be complementary, such as at least 90% complementary, such as at least 95% complementary to an RNA region comprising a sequence shown in SEQ ID NO: 10.

SEQ ID NO: 10:
CAGCAGCAGCAGCAGCAGCAGCAGCAGCAGCAGCAGCAG

Alternatively, the antisense oligonucleotide, or contiguous nucleotide sequence, may be complementary, such as at least 90% complementary, such as at least 95% complementary to an RNA region comprising a sequence shown in SEQ ID NO: 11.

SEQ ID NO: 11:
CAGCAGCAGCAGCAGCAGCAGCAGCAGCAGCAGCAGCAGC
AGCAGCAGCAGCAGCAG

Moreover, the antisense oligonucleotide, or contiguous nucleotide sequence, may be complementary, such as at least 90% complementary, such as at least 95% complementary to an RNA region comprising a sequence shown in SEQ ID NO: 12.

SEQ ID NO: 12:
CAGCAGCAGCAGCAGCAGCAGCAGCAGCAGCAGCAGCAGCAGCAGCAG
CAGCAGCAGCAGCAGCAGCAG

Thus, the antisense oligonucleotide may comprise a contiguous nucleotide sequence which is complementary, such as at least 90% complementary, such as at least 95% complementary to an RNA region comprising SEQ ID NO: 10, 11, or 12.

In an embodiment, the complementarity is over the entire length of the target sequence (SEQ ID NO; 10, 11, or 12).

It is to be understood that the target sequence shall be within a transcribed portion of an mRNA encoding a polyQ disease-related protein.

### Compound

Herein, the term “compound” means any antisense oligonucleotide being capable of specifically binding to a CAG repeat region of an mRNA encoding a polyQ disease-related protein such that a double-stranded RNA is formed which is capable of attracting an Adenosine Deaminase Acting on RNA (ADAR) inserting an A to I exchange into at least one CAG trinucleotide of the CAG repeat region of said mRNA. For example, the compound may be an antisense RNA oligonucleotide.

### Olignonucleotide

The term “oligonucleotide” (herein also referred to as “nucleic acid molecule”) as used herein is defined as it is generally understood by the skilled person as a molecule comprising two or more covalently linked nucleosides. Such covalently bound nucleosides may also be referred to as nucleic acid molecules or oligomers. The oligonucleotides referred to in the description and claims may be therapeutic oligonucleotides below 200 nucleotides in length. In an embodiment, the oligonucleotide or contiguous nucleotide sequence as referred to herein has a length of at least 20, at least 25, at least 30, at least 40, at least 45, or at least 50 nucleotides.

In an embodiment, the oligonucleotide or contiguous nucleotide sequence has a length of 20 to 150 nucleotides.

In an alternative embodiment, the oligonucleotide or contiguous nucleotide sequence has a length of 25 to 150 nucleotides.

In an alternative embodiment, the oligonucleotide or contiguous nucleotide sequence has a length of 30 to 150 nucleotides.

In an alternative embodiment, the oligonucleotide or contiguous nucleotide sequence has a length of 25 to 100 nucleotides.

In an alternative embodiment, the oligonucleotide or contiguous nucleotide sequence has a length of 30 to 100 nucleotides.

In an alternative embodiment, the oligonucleotide or contiguous nucleotide sequence has a length of 40 to 100 nucleotides.

In an alternative embodiment, the oligonucleotide or contiguous nucleotide sequence has a length of 40 to 70 nucleotides.

In an alternative embodiment, the oligonucleotide or contiguous nucleotide sequence has a length of 40 to 70 nucleotides.

The oligonucleotide may be or comprise a single stranded antisense oligonucleotide, such as a single stranded RNA antisense oligonucleotide. Therapeutic oligonucleotide molecules are commonly made in the laboratory by solid-phase chemical synthesis followed by purification and isolation. The ASO may be also delivered to cells using vector, such as viral vectors (such as lentiviral or adenoviral vectors from which they are then transcribed to produce the RNA antisense oligonucleotide. In an embodiment of the present invention, the ASO is a chemically produced RNA antisense oligonucleotide (not relying on cell-based expression from plasmids or viruses).

When referring to a sequence of the oligonucleotide, reference is made to the sequence or order of nucleobase moieties, or modifications thereof, of the covalently linked nucleotides or nucleosides. Generally, the oligonucleotide of the invention is man-made, and is chemically synthesized, and is typically purified or isolated. In some embodiments, the oligonucleotide of the invention is a RNA ASO transcribed from a vector upon entry into the target cell. The oligonucleotide of the invention may comprise one or more modified nucleosides or nucleotides. Accordingly, the present invention also pertains to a vector comprising a polynucleotide encoding the RNA antisense oligonucleotide of the invention. In an embodiment, the polynucleotide is operably linked to a promoter which allows for expressing said RNA antisense oligonucleotide. Such vector may be a viral vector, such as an adenoviral vector.

In some embodiments, the oligonucleotide of the invention may comprise one or more modified nucleosides or nucleotides, such as 2′ sugar modified nucleosides. In some embodiments, the oligonucleotide comprises phosphorothioate internucleoside linkages. In some embodiments, the oligonucleotide may be conjugated to non-nucleosidic moieties (conjugate moieties).

### Antisense Oligonucleotides

The term “antisense oligonucleotide”, “ASO” or “nucleic acid molecule” as used herein is defined as oligonucleotide that is capable of specifically binding, i.e. hybridizing, to a CAG repeat region of an mRNA encoding a polyQ disease-related protein such that a double-stranded RNA is formed. The first strand of the double-stranded RNA is the mRNA, the second strand is the ASO. Said double-stranded RNA is capable of attracting an Adenosine Deaminase Acting on RNA (ADAR) polypeptide inserting an A to I exchange into at least one CAG trinucleotide of the CAG repeat region of said mRNA. Accordingly, the formed double-stranded RNA shall allow for the hydrolytic deamination of adenosine to inosine in at least one CAG trinucleotide of the CAG repeat region of said mRNA by ADAR.

Preferably, the antisense oligonucleotide of the present invention is not essentially double-stranded and is therefore not an RNAi molecule, such as a siRNA or short hairpin RNA (shRNA). Preferably, the antisense oligonucleotide of the present invention is single-stranded. As used herein, the term “single-stranded” means that the oligonucleotide lacks sufficient self-complementarity to form a stable self-duplex It is understood that single stranded oligonucleotides of the present invention can form intermolecular duplex structures (duplex between two molecules of the same oligonucleotide), as long as the degree of intra or inter self complementarity is less than 50%, such as less than 30% or less than 20% across of the full length of the oligonucleotide.

As set forth above, the ASO of the present invention is not an RNAi molecule. Herein, the term “RNA interference (RNAi) molecule” refers to short double-stranded oligonucleotide containing RNA nucleosides and which mediates targeted cleavage of an RNA transcript via the RNA-induced silencing complex (RISC), where they interact with the catalytic RISC component argonaute. The RNAi molecule modulates, e g., inhibits, the expression of the target nucleic acid in a cell, e.g. a cell within a subject, such as a mammalian subject. RNAi molecules include single-stranded RNAi molecules (Lima at al 2012 Cell 150:883) and double stranded siRNAs, as well as short hairpin RNAs (shRNAs).

In some embodiments of the invention, the oligonucleotide of the invention is not an RNAi agent, such as a siRNA. The term “small interfering ribonucleic acid” or “siRNA” refers to a small interfering ribonucleic acid RNAi molecule. It is a class of double-stranded RNA molecules, also known in the art as short interfering RNA or silencing RNA.

Preferably, the antisense oligonucleotide of the present invention is unphosphorylated at the 5′ end.

Advantageously, the antisense oligonucleotide of the present invention in RNA antisense oligonucleotide.

Advantageously, the oligonucleotide of the invention comprises one or more modified nucleosides or nucleotides, such as 2′ sugar modified nucleosides.

### Double Stranded RNA

The antisense oligonucleotide of the present invention shall be capable of binding to a CAG repeat region of an mRNA encoding a polyQ disease-related protein such that a double stranded RNA is formed which is capable of attracting to Adenosine Deaminase Acting on RNA (ADAR) as described elsewhere herein.

As described elsewhere herein, the antisense RNA oligonucleotide of the present invention may comprise modified nucleosides. In accordance with the present invention, Nucleosides with modifications are still considered as nucleosides if they allow Watson Crick base pairing. Further, ADAR would be still active even if some of the nucleosides in the doubled-stranded structure are DNA nucleosides. The “RNA antisense oligonucleotide” as referred to herein may comprise some DNA nucleosides. The DNA nucleosides may be chemically modified or unmodified. In some embodiments, up to 70% nucleosides of the oligonucleotide, or contiguous nucleotide sequence thereof, are DNA nucleosides, such as up to 60%, such as up to 50%, such as up to 40%, such as up to 30% or such as up to 20% of the nucleosides in the oligonucleotide, or contiguous nucleotide sequence thereof, are DNA nucleosides. In some embodiments, all of the nucleosides of the oligonucleotide, or contiguous nucleotide sequence thereof, are RNA nucleosides.

Since some DNA nucleosides may be present, the term “double stranded RNA” also encompasses double-stranded complexes in which one strand (the mRNA) consists of RNA and one strand comprises at least portion DNA nucleosides. However, the formed dsRNA shall be capable of attracting an Adenosine Deaminase Acting on RNA (ADAR) that introduces an A to I exchange into at least one CAG trinucleotide of the CAG repeat region of said mRNA.

For doing so, the formed dsRNA has to have a minimal length. This is well known in the art and understood by the skilled person.

The length of the formed dsRNA, i.e. the formed dsRNA complex, may depend on the length of the ASO. In an embodiment, it has a length of at least 20, at least 25, at least 30, at least 40, at least 45, or at least 50 base pairs (bp).

In an embodiment, the formed dsRNA has a length of 20 to 150 bp.

In an alternative embodiment, the formed dsRNA has a length of 25 to 150 bp.

In an alternative embodiment, the formed dsRNA has a length of 30 to 150 bp.

In an alternative embodiment, the formed dsRNA has a length of 25 to 100 bp.

In an alternative embodiment, the formed dsRNA has a length of 30 to 100 bp.

In an alternative embodiment, the formed dsRNA has a length of 40 to 100 bp.

In an alternative embodiment, the formed dsRNA has a length of 40 to 70 bp.

In an alternative embodiment, the formed dsRNA has a length of 40 to 70 bp.

### Contiguous Nucleotide Sequence

The term “contiguous nucleotide sequence” refers to the region of the nucleic acid molecule which is complementary to the target nucleic acid, i.e. the CAG repeat region. The term is used interchangeably herein with the term “contiguous nucleobase sequence” and the term “oligonucleotide motif sequence”. In some embodiments, all the nucleotides of the oligonucleotide constitute the contiguous nucleotide sequence. In some embodiments, the ASO may optionally comprise further nucleotide(s), for example a nucleotide linker region which may be used to attach a functional group (e.g. a conjugate group for targeting) to the contiguous nucleotide sequence. The nucleotide linker region may or may not be complementary to the target nucleic acid. In some embodiments, the nucleobase sequence of the antisense oligonucleotide is the contiguous nucleotide sequence.

### Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo) base moiety. Advantageously, one or more of the modified nucleoside comprises a modified sugar moiety. The term “modified nucleoside” may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

### Modified Internucleoside Linkage

The term “modified internucleoside linkage” is defined as generally understood by the skilled person as linkages other than phosphodiester (PO) linkages, that covalently couples two nucleosides together. The oligonucleotides of the invention may therefore comprise one or more modified internucleoside linkages, such as one or more phosphorothioate internucleoside linkages, or one or more phosphorodithioate internucleoside linkages. With the oligonucleotide of the invention it is advantageous to use phosphorothioate internucleoside linkages.

Phosphorothioate internucleoside linkages are particularly useful due to nuclease resistance, beneficial pharmacokinetics and ease of manufacture. In some embodiments, at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments, all of the internucleoside linkages of the oligonucleotide, or contiguous nucleotide sequence thereof, are phosphorothioate.

In some advantageous embodiments, all the internucleoside linkages of the contiguous nucleotide sequence of the oligonucleotide are phosphorothioate, or all the internucleoside linkages of the oligonucleotide are phosphorothioate linkages.

It is recognized that, as disclosed in EP 2 742 135, antisense oligonucleotides may comprise other internucleoside linkages (other than phosphodiester and phosphorothioate), for example alkyl phosphonate/methyl phosphonate internucleoside linkages, which according to EP 2 742 135 may for example be tolerated in an otherwise DNA phosphorothioate gap region.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

The terms “adenine”, “guanine”, “cytosine”, “thymine”, “uracil” and ‘hypoxanthine” (which is the nucleobase in inosine) shall refer to the nucleobases as such. The terms “adenosine”, “guanosine”, “cytidine”, “thymidine”, “uridine” and “inosine”, shall refer to the nucleobases linked to the ribosyl sugar. However, the terms adenosine and adenine, guanosine and guanine, cytosine and cytidine, uracil and uridine, thymine and thymidine, inosine and hypoxanthine, are also used interchangeably herein to refer to the corresponding nucleobase, nucleoside or nucleotide.

The nucleobase moieties may be indicated by the letter code for each corresponding nucleobase, e.g. A, T, G, C, U or I, wherein each letter may optionally include modified nucleobases of equivalent function.

In some embodiments, the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′ thio-thymine, inosine, diaminopurine, 6-aminopurine, 2-aminopurine, 2,6-diaminopurine and 2-chloro-6-aminopurine.

### Complementarity

As set forth elsewhere herein, the ASO of the present invention shall be capable of specifically binding, i.e. hybridizing, to a CAG repeat region of an mRNA encoding a polyQ disease-related protein such that a double-stranded RNA is formed. Thus, the ASO shall be complementary to CAG region.

The term “complementarity” or “complementary” describes the capacity for Watson-Crick base-pairing of nucleosides/nucleotides. Watson-Crick base pairs are guanine (G)-cytosine (C) and adenine (A)-thymine (T)/uracil (U). Further, inosine (I) pairs with cytosine (C) in a Watson-Crick bonding configuration.

It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity). Further, inosine is considered to be identical to a guanine for the purpose of calculating % identity.

In order to allow for an A to I editing, i.e. an A to I exchange, in the target mRNA by ADAR, it is required that the contiguous nucleotide sequence of the ASO of the present invention and the target sequence are not fully complementary. Thus, there shall be at least one mismatch between the contiguous nucleotide sequence and the target sequence. However, it is envisaged that that contiguous nucleotide sequence has at least 80%, such as at least 85 least 90%, at least 91%, at least 92%, at least 93%, at least 94%, at least 95%, at least 96%, at least 97%, at least 98% complementarity to the target sequence. In some embodiments, the contiguous nucleotide sequence is fully complementary to the target sequence.

However, it is also envisaged that the ASO or contiguous nucleotide sequence comprises less than 100% complementarity to the target sequence. Specifically, it is envisaged that the ASO, or contiguous nucleotide sequence, comprises at least one mismatch to at least one adenosine (i.e. target inosine) in the CAG repeat region (i.e. the target region) of an mRNA encoding a polyQ disease-related protein. In other words, the ASO of the present invention typically comprises at least one mismatch with a target adenosine within a CAG repeat. However, the ASO shall be still capable of binding, i.e. hybridizing to the target sequence. The CAG repeat region is the region in the mRNA encoding the polyQ tract. The target adenosine is the middle nucleotide (A) in a CAG trinucleotide.

In an embodiment, the nucleoside(s) in the antisense oligonucleotide of the present invention which is (are) mismatched with a target adenosine present in a CAG trinucleotide may be any nucleoside except Uracil. In an embodiment, said nucleoside(s) may be selected from Cytosine, Adenine and Guanine. Preferably, said nucleoside(s) is (are) Cytosine(s). Thus, the one or mismatched nucleotides in the antisense oligonucleotide (opposite to the target adenosine) may be Cytosine(s). In cases in which the antisense oligonucleotide of the present invention is fully commentary to the target sequence, an Uracil may be present.

In an embodiment, the ASO or contiguous nucleotide sequence comprises at least two mismatches with at least two adenosines in the CAG repeat region.

In an embodiment, the ASO or contiguous nucleotide sequence comprises at least three mismatches with at least three adenosines in the CAG repeat region.

In an embodiment, the ASO or contiguous nucleotide sequence comprises at least four mismatches with at least four adenosines in the CAG repeat region.

In an embodiment, the ASO or contiguous nucleotide sequence comprises at least five mismatches with at least five adenosines in the CAG repeat region.

Further, it is envisaged that the ASO or contiguous nucleotide sequence comprises 1 to 15, such as 1 to 10, such as 2 to 10, such as 3 to 10, such as 3 to 8 mismatches with a target adenosine within CAG trinucleotides present in the CAG repeat region.

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95:1460-1465 using appropriately derived thermodynamic parameters described by Sugimoto et al., 1995, Biochemistry 34:11211-11216 and McTigue et al., 2004, Biochemistry 43:5388-5405. In some embodiments, the degree or strength of hybridization is measured by the standard state Gibbs free energy ΔG°.

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Target Cell

The term a “target cell” as used herein refers to a cell which is expressing the target nucleic acid. For the therapeutic use of the present invention, it is advantageous if the target cell is a cell which expresses a polyQ disease-related protein, such as the ATXN3 polypeptide. In some embodiments, the target cell may be in vivo or in vitro. In some embodiments, the target cell is a mammalian cell such as a rodent cell, such as a mouse cell or a rat cell, or a woodchuck cell or a primate cell such as a monkey cell (e.g. a cynomolgus monkey cell). In some embodiments, the cell is a human cell . . .

In an embodiment, the target cell may be a brain cell or another cell of the CNS (central nervous system), such as a cell of the spinal cord. Thus, the target cell may be a cell of the central nervous system.

In another embodiment, the target cell may be a heart cell, such as a cell in the myocardium.

In another embodiment, the target cell may be a muscle cell.

The target cell shall comprise a polypeptide having ADAR activity. In an embodiment, said polypeptide is the endogenous ADAR enzyme. In another embodiment, said polypeptide is recombinantly expressed in the target cell. In yet another embodiment, said polypeptide has been delivered to the target cell.

### Effect of the Antisense Oligonucleotide of the Present Invention

The oligonucleotide of the invention shall reduce the expression of polyQ disease-associated proteins having a pathogenic (i.e. disease causing) length of the polyQ tract. This is achieved by converting one or more CAG trinucleotides to CGG trinucleotides on the mRNA level. This results in in one or more interruptions of the polyQ tracts by arginine. Thus, polyQ disease-associated polypeptides are expressed which comprise one or more arginine residues in the polyQ tract.

The term “inhibition of expression” or “reducing expression” as used herein is to be understood as an overall term for the reduction of the amount of polyQ disease-associated proteins having a pathogenic (i.e. disease causing) length of the polyQ tract in a target cell.

Inhibition of expression or activity may be determined by measuring the level of mRNA encoding said polyQ disease-associated protein, or by measuring the level of the polyQ disease-associated protein. Inhibition of expression may be determined in vitro or in vivo. Advantageously, the inhibition is assessed in relation to the amount of the polyQ disease associated polypeptide before administration of the oligonucleotide of the present invention. Alternatively, inhibition is determined by reference to a control. It is generally understood that the control is an individual or target cell treated with a saline composition or an individual or target cell treated with a non-targeting oligonucleotide (mock).

In some embodiments, the amount of polyQ disease-associated protein with a disease causing length of the polyQ tract is decreased as compared to a control. In some embodiments, the decrease in amount is at least 20%, such as at least 30%, such as at least 40% as compared to a control.

### Modified Oligonucleotide

The term “modified oligonucleotide” describes an oligonucleotide comprising one or more sugar-modified nucleosides and/or modified internucleoside linkages. The term “chimeric” oligonucleotide is a term that has been used in the literature to describe oligonucleotides comprising modified nucleosides. The antisense oligonucleotide of the invention is advantageously a chimeric oligonucleotide.

### Sugar Modifications

The oligonucleotide of the invention may comprise one or more nucleosides which have a modified sugar moiety, i.e. a modification of the sugar moiety when compared to the ribose sugar moiety found in DNA and, in particular RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

### High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably results in an increase in melting temperature in the range of +0.5 to +12° C., more preferably in the range of +1.5 to +10° C. and most preferably in the range of +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3 (2), 293-213).

### 2′ Sugar Modified Nucleosides

A 2′ sugar modified nucleoside is a nucleoside which has a substituent other than H or —OH at the 2′ position (2′ substituted nucleoside) or comprises a 2′ linked biradical capable of forming a bridge between the 2′ carbon and a second carbon in the ribose ring, such as LNA (2′-4′ biradical bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3 (2), 293-213, and Deleavey and Damha, Chemistry and Biology 2012, 19, 937. Below are illustrations of some 2′ substituted modified nucleosides.

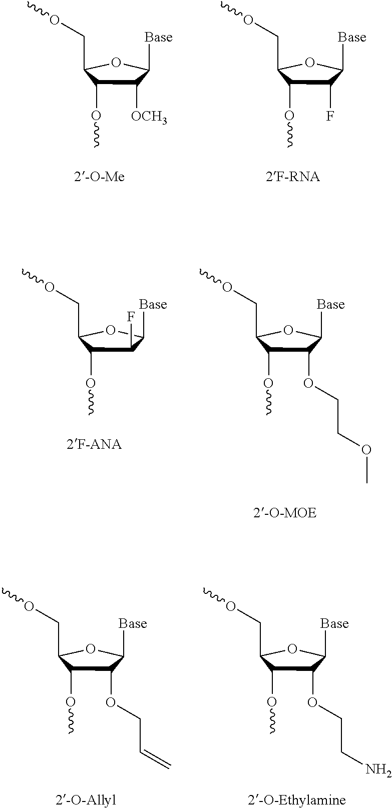

In relation to the present invention 2′ substituted sugar modified nucleosides does not include 2′ bridged nucleosides like LNA.

### Locked Nucleic Acid Nucleosides (LNA Nucleoside)

A “LNA nucleoside” is a 2′-sugar modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75 (5) pp. 1569-81, Mitsuoka et al., Nucleic Acids Research 2009, 37 (4), 1225-1238, and Wan and Seth, J. Medical Chemistry 2016, 59, 9645-9667.

Particular examples of LNA nucleosides of the invention are presented in Scheme 1 (wherein B is as defined above).

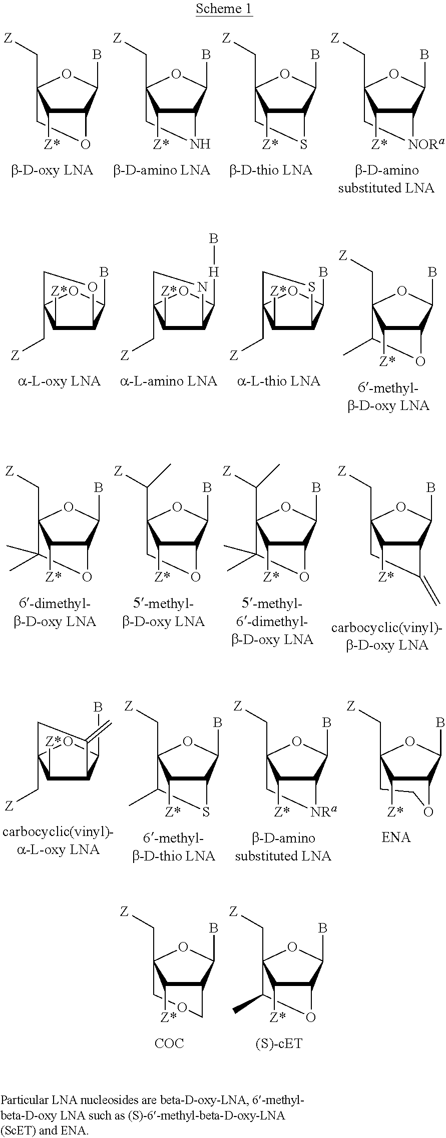

### Conjugate

For biological distribution, the oligonucleotide as set forth herein may be conjugated to a targeting ligand, and/or be formulated into lipid nanoparticles. In an example, the nucleic acid molecule is conjugated to a moiety that targets a brain cell or another cell of the CNS. Thus, the nucleic acid molecule may be conjugated to a moiety that facilitates delivery across the blood brain barrier. For example, the nucleic acid molecule may be conjugated to an antibody or antibody fragment targeting the transferrin receptor, such as the human transferrin receptor.

The term conjugate as used herein refers to an oligonucleotide which is covalently linked to a non-nucleotide moiety. The conjugate moiety may be covalently linked to the antisense oligonucleotide, optionally via a linker.

Oligonucleotide conjugates and their synthesis have been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103, each of which is incorporated herein by reference in its entirety.

### Treatment

The term “treatment” as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic. Prophylactic can be understood as preventing that the disease becomes symptomatic.

### Patient

For the purposes of the present invention the “subject” (or “patient”) may be a vertebrate. In context of the present invention, the term “subject” includes both humans and other animals, particularly mammals, and other organisms. Thus, the herein provided means and methods are applicable to both human therapy and veterinary applications. Preferably, the subject is a mammal. More preferably, the subject is human.

As described elsewhere herein, the patient to be treated shall suffer from a polyQ disease, such as from SCA3, or shall be at risk of suffering from a polyQ disease. Thus, it is envisaged that the patient to be treated comprises at least one gene encoding for a polyQ disease-related protein having a pathogenic (i.e. disease causing) length.

### DETAILED DESCRIPTION OF THE INVENTION

The present invention relates to an antisense oligonucleotide, wherein said antisense oligonucleotide is capable of specifically binding to a CAG repeat region of an mRNA encoding a polyQ disease-related protein such that a double stranded RNA is formed which is capable of attracting an Adenosine Deaminase Acting on RNA (ADAR) inserting an A to I exchange into at least one CAG trinucleotide of the CAG repeat region of said mRNA.

In an embodiment, said antisense oligonucleotide is an RNA.

In an embodiment, said antisense oligonucleotide is a single-stranded antisense oligonucleotide, such as a single-stranded RNA.

In an embodiment, said antisense oligonucleotide has a length of 20 to 150 nucleotides, such as 25 to 100 nucleotides, such as 40 to 70 nucleotides.

In an embodiment, said antisense oligonucleotide comprises at least nine CUG trinucleotides and at least one CCG trinucleotide and/at least one CCI trinucleotide, such as at least two, three, four, five, six, seven, or eight CCG trinucleotides and/or CCI trinucleotides. Typically, the antisense oligonucleotide does not comprise intervening sequences between the CUG, CCI and CCG trinucleotides. However, it is envisaged, that the oligonucleotide comprises one or more mismatches to the target nucleic acid, such as one or more mismatches to one or more adenosines present in the CAG region.

Typically, the antisense oligonucleotide of the present invention. or the contiguous nucleotide sequence as set forth herein, comprises the following sequence:

(SEQ ID NO: 13)
CX₁X₂CX₃X₄CX₅X₆CX₇X₈CX₉X₁₀CX₁₁X₁₂CX₁₃X₁₄CX₁₅X₁₆CX₁₇X₁₈CX₁₉X₂₀,

wherein X₁, X₃, X₅, X₇, X₉, X₁₁, X₁₃, X₁₅, X₁₇ and X₁₉ are independently Uracil or Cytosine,
wherein at least one of X₁, X₃, X₅, X₇, X₉, X₁₁, X₁₃, X₁₅, X₁₇ and X₁₉ is Cytosine, and
wherein X₂, X₄, X₆, X₈, X₁₀, X₁₂, X₁₄, X₁₆, X₁₈ and X₂₀ are independently Guanine or Inosine.

For example, one, two, three, four, or five nucleotides of X1, X3, X5, X7, X9, X11, X13, X15, X17 and X19 is (are) Cytosine(s).

In some embodiments, one nucleotide of X1, X3, X5, X7, X9, X11, X13, X15, X17 and X19 is Cytosine.

In some embodiments, two nucleotides of X1, X3, X5, X7, X9, X11, X13, X15, X17 and X19 are Cytosines.

In some embodiments, three nucleotides of X1, X3, X5, X7, X9, X11, X13, X15, X17 and X19 are Cytosines.

In some embodiments, four nucleotides of X1, X3, X5, X7, X9, X11, X13, X15, X17 and X19 are Cytosines.

In some embodiments, five nucleotides of X1, X3, X5, X7, X9, X11, X13, X15, X17 and X19 are Cytosines.

In an embodiment, at least one nucleotide, such as one nucleotide, or two, three, four or five nucleotides of X₂, X₄, X₆, X₈, X₁₀, X₁₂, X₁₄, X₁₆, X₁₈ and X₂₀ is (are) Inosine(s). Preferably, said inosine residue(s) is (are) are positioned 3′ to a Cytosine residue.

SEQ ID NO: 13 has a length of 30 nucleotides. It is to be understood that antisense oligonucleotide of the present invention, or the contiguous nucleotide sequence as set forth herein, may be longer than 30 nucleotides (as described elsewhere herein).

In a preferred embodiment, the antisense oligonucleotide comprises at least one 2′ sugar modified nucleoside. Typically, said at least one 2′ sugar modified nucleoside is selected from the group consisting of: 2′-O-alkyl-RNA, 2′-O-methyl-RNA, 2′-O-alkoxy-RNA, 2′-O-methoxyethyl-RNA, 2′-fluoro-RNA, arabino nucleic acid (ANA), 2′-fluoro-ANA, and an LNA nucleoside.

For example, at least one 2′ sugar modified nucleoside may be 2′-O-methyl-RNA.

Preferably, the antisense oligonucleotide of the present invention comprises at least one phosphorothioate internucleoside linkage. For example, 10-90%, such as 20-80%, such as 30-70%, such as 40-60% of internucleoside linkages in the antisense oligonucleotide are phosphorothioate internucleoside linkages. All other linkages could be phosphodiester internucleoside linkages.

In an embodiment, all internucleoside linkages in the antisense oligonucleotide (and thus 100%) are phosphorothioate internucleoside linkages.

In an embodiment, said antisense oligonucleotide comprises or consists of a nucleic acid sequence as shown in SEQ ID NO: 1.

In another embodiment, said antisense oligonucleotide comprises or consists of a nucleic acid sequence as shown in SEQ ID NO: 2.

In another embodiment, said antisense oligonucleotide comprises or consists of a nucleic acid sequence as shown in SEQ ID NO: 3.

In another embodiment, said antisense oligonucleotide comprises or consists of a nucleic acid sequence as shown in SEQ ID NO: 4.

In another embodiment, said antisense oligonucleotide comprises or consists of a nucleic acid sequence as shown in SEQ ID NO: 5.

In another embodiment, said antisense oligonucleotide comprises or consists of a nucleic acid sequence as shown in SEQ ID NO: 6.

For example, the oligonucleotide comprises or consists of a contiguous nucleotide sequence, selected from the group consisting of:

(SEQ ID NO: 1)
g*c*u*g*c* U*G*C*U*G*C*U*G*C*U*G*C*U*G*C*U*G* CUGCUGCUGCUG CCG
CUGCUGCUG*C* u*g*c*u*g,
(SEQ ID NO: 2)
g*c*u*g*c*U*G*C*U*G*C*U*G*C*U*G*C*U*G*C*U*G* CUGCUGCUGCUG CCI
CUGCUGCUG*C* u*g*c*u*g,
(SEQ ID NO: 3)
g*c*u*g*c* U*G*C*U*G*C*U*G*C*U*G*C*U*G*C*U*G*CUGCUGCUG CCI CCI
CUGCUGCUG*C* u*g*c*u*g,
(SEQ ID NO: 4)
g*c*u*g*c* U*G*C*U*G*C*U*G*C*U*G*C*U*G*C*U*G*CUGCUG CCI CUG CCI
CUGCUGCUG*C* u*g*c*u*g,
(SEQ ID NO: 5)
g*c*u*g*c* U*I*C*U*G*C*U*I*C*U*G*C*U*I*C*U*G*CUGCUG CCI CUG CCI
CUGCUG*C*U*I*C* U*G*C*U*I* C*U*G*C*U*I*C*U* g*c*u*g*c,
or
(SEQ ID NO: 6)
g*c*u*g*c* U*G*C*U*G*C*U*G*C*U*G*C*U*G*C*U*G* CCI CUG CCI CUG CCI CUG
CCI* C*U*G*C* U*G*C*U*G* C*U*G*C*U*G*C*U* g*c*u*g*c

wherein a capital letter represents a RNA nucleoside, a lower case letter represents a 2′-O-alkyl-RNA nucleoside, such as 2′-O-methyl-RNA nucleoside, and wherein an asterisk (*) represents a phosphorothioate internucleoside linkage, and wherein all other linkages are phosphodiester internucleoside linkages.

The antisense oligonucleotide of the present invention can be used in the treatment of a polyQ disease. In an embodiment, the polyQ disease is selected from the group consisting of: Huntington's disease (HD), Spinocerebellar ataxia (SCA) 1, 2, 3, 6, 7, or 17, Dentatorubralpallidoluysian atrophy, and Kennedy's disease. For example, the polyQ disease may be SCA3.

In an embodiment, the polyQ disease is HD and the polyQ disease-related protein is HTT.

In another embodiment, the polyQ disease is SCA 1 and the polyQ disease-related protein is ATXN1.

In another embodiment, the polyQ disease is SCA 2 and the polyQ disease-related protein is ATXN2.

In another embodiment, the polyQ disease is SCA 3 and the polyQ disease-related protein is ATXN3.

In another embodiment, the polyQ disease is SCA 6 and the polyQ disease-related protein is CACNA1A.

In another embodiment, the polyQ disease is SCA 7 and the polyQ disease-related protein is ATXN7.

In another embodiment, the polyQ disease is SCA 12 and the polyQ disease-related protein is PPP2R2B.

In another embodiment, the polyQ disease is SCA 17 and the polyQ disease-related protein is TBP.

In another embodiment, the polyQ disease is Dentatorubralpallidoluysian atrophy and the polyQ disease-related protein is ATN1 or DRPLA.

In another embodiment, the polyQ disease is Kennedy's disease and the polyQ disease-related protein is AR.

In accordance with the present invention, it is envisaged that the polyQ disease-related protein shall comprise a polyQ tract which has a disease-causing length, i.e. a pathogenic length.

Typically, the mRNA encoding the HTT protein comprises 36 to 250 consecutive CAG repeats.

Typically, the mRNA encoding the ATXN1 protein comprises 49 to 88 consecutive CAG repeats.

Typically, the mRNA encoding the ATXN2 protein comprises 32 to 100 consecutive CAG repeats.

Typically, the mRNA encoding the ATXN3 protein comprises 55 to 86 consecutive CAG repeats.

Typically, the mRNA encoding the CACNA1A protein comprises 49 to 88 consecutive CAG repeats.

Typically, the mRNA encoding the ATXN7 protein comprises 37 to 306 consecutive CAG repeats.

Typically, the mRNA encoding the PPP2R2B protein comprises 55 to 78 consecutive CAG repeats.

Typically, the mRNA encoding the TBP protein comprises 47 to 63 consecutive CAG repeats protein comprises 49 to 88 consecutive CAG repeats.

Typically, the mRNA encoding the ATN1 protein comprises 49 to 88 consecutive CAG repeats.

Typically, the mRNA encoding the AR protein comprises 38 to 62 consecutive CAG repeats.

In an embodiment, the antisense oligonucleotide of the present invention reduces or prevents aggregation of the polyQ disease-related protein. Preferably, it is prevented by inserting an A to I exchange into at least one CAG trinucleotide of the CAG repeat region of the mRNA of the polyQ disease-related protein.

The present invention further pertains to a polyQ disease-associated polypeptide comprising one or more arginine residues in its polyQ tract. The term “polyQ disease-associated polypeptide” has been described above. In an embodiment, the polyQ disease-associated polypeptide shall comprise a polyQ tract having a pathogenic, i.e. disease causing length as described elsewhere herein. However, the polyQ tract of the polyQ disease-associated polypeptide shall be interrupted with one or more arginine residues.

In an embodiment, the polyQ disease-associated polypeptide comprises a polyQ tract which comprises, i.e. is interrupted by, at least one arginine residue.

In an embodiment, the polyQ disease-associated polypeptide comprises a polyQ tract which comprises, i.e. is interrupted by, at least two arginine residues.

In an embodiment, the polyQ disease-associated polypeptide comprises a polyQ tract which comprises, i.e. is interrupted by, at least three arginine residues.

In an embodiment, the polyQ disease-associated polypeptide comprises a polyQ tract which comprises, i.e. is interrupted by, at least four arginine residues.

In an embodiment, the polyQ disease-associated polypeptide comprises a polyQ tract which comprises, i.e. is interrupted by, at least five arginine residues.

In some embodiments, the polypeptide of the present invention is an isolated polypeptide. Said polypeptide may be also produced recombinant means, e.g. for research studies.

The present invention also relates to a polynucleotide encoding the polypeptide of the present invention. The polynucleotide may be operably linked to a promoter, such as a heterologous promoter.

### Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand) to covalently attach the conjugate moiety to the oligonucleotide. In a further aspect, a method is provided for manufacturing the composition of the invention, comprising mixing the oligonucleotide or conjugated oligonucleotide of the invention with a pharmaceutically acceptable diluent, solvent, carrier, salt and/or adjuvant.

### Pharmaceutical Salt

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of the present invention.

In a further aspect, the invention provides a pharmaceutically acceptable salt of the nucleic acid molecules or a conjugate thereof, such as a pharmaceutically acceptable sodium salt, ammonium salt or potassium salt.

### Pharmaceutical Composition

In a further aspect, the invention provides pharmaceutical compositions comprising any of the compounds of the invention, in particular the aforementioned nucleic acid molecules, i.e. antisense oligonucleotides and/or nucleic acid molecule conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments, the pharmaceutically acceptable diluent is sterile phosphate buffered saline.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

In some embodiments, the nucleic acid molecule or the nucleic acid molecule conjugates of the invention, or pharmaceutically acceptable salt thereof is in a solid form, such as a powder, such as a lyophilized powder.

Compounds, nucleic acid molecules or nucleic acid molecule conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents.

In some embodiments, the nucleic acid molecule or nucleic acid molecule conjugate of the invention is a prodrug. In particular with respect to nucleic acid molecule conjugates the conjugate moiety is cleaved off the nucleic acid molecule once the prodrug is delivered to the site of action, e.g. the target cell.

### Administration

The nucleic acid molecules or nucleic acid molecule conjugates or pharmaceutical compositions of the present invention may be administered enteral (such as, orally or through the gastrointestinal tract) or parenteral (such as, intravenous, subcutaneous, intramuscular, intracerebral, intracerebroventricular or intrathecal).

In a preferred embodiment, the oligonucleotide or pharmaceutical compositions of the present invention are administered by a parenteral route including intravenous, intraarterial, subcutaneous, intraperitoneal or intramuscular injection or infusion. In one embodiment, the active nucleic acid molecule or nucleic acid molecule conjugate is administered intravenously. In another embodiment, the active nucleic acid molecule or nucleic acid molecule conjugate is administered subcutaneously.

The invention also provides for the use of the nucleic acid molecule or nucleic acid molecule conjugate of the invention as described for the manufacture of a medicament wherein the medicament is in a dosage form for subcutaneous administration.

### Applications

The nucleic acid molecules of the invention may be utilized as research reagents for, for example, diagnostics, therapeutics and prophylaxis.

In research, such nucleic acid molecules may be used to specifically modulate the synthesis of polyQ disease-related proteins in cells (e.g. in vitro cell cultures) and experimental animals thereby facilitating functional analysis of the target or an appraisal of its usefulness as a target for therapeutic intervention.

Also encompassed by the present invention is an in vivo or in vitro method for modulating polyQ disease-related protein expression in a target cell which is expressing a polyQ disease-related protein, said method comprising administering a nucleic acid molecule, conjugate compound or pharmaceutical composition of the invention in an effective amount to said cell.

In some embodiments, the target cell, is a mammalian cell in particular a human cell. The target cell may be an in vitro cell culture or an in vivo cell forming part of a tissue in a mammal. In preferred embodiments, the target cell is present in in the brain or the spinal cord.

One aspect of the present invention is related to the nucleic acid molecules, conjugate compounds or pharmaceutical compositions of the invention for use as a medicament.

In an aspect of the invention, the nucleic acid molecule, conjugate compound or pharmaceutical composition of the invention is capable of reducing the level of a polyQ disease-related protein having a pathogenic (i.e. disease causing) length of the polyQ tract. This is achieved by generating polyQ disease-associated polypeptides comprising one or more arginine residues in its polyQ tract.

For example, the nucleic acid molecule may reduce the level of polyQ disease-related protein having a pathogenic (i.e. disease causing) length of the polyQ tract in a cell by at least 20%, at least 40% such as 50% or 60%, reduction compared to controls. The controls may be untreated cells or animals, or cells or animals treated with an appropriate negative control.

Accordingly, one aspect of the present invention is related to use of the nucleic acid molecule, conjugate compounds or pharmaceutical compositions of the invention to reduce the amount of polyQ disease-related protein in a subject.

A further aspect of the invention relates to the use of the nucleic acid molecules, conjugate compounds or pharmaceutical compositions of the invention to treat a polyQ disease as set forth herein.

A further aspect of the invention relates to the use of the nucleic acid molecules, conjugate compounds or pharmaceutical compositions of the invention to inhibit development of symptomatic polyQ disease.

The subject to be treated with the nucleic acid molecules, conjugate compounds or pharmaceutical compositions of the invention (or which prophylactically receives nucleic acid molecules, conjugate compounds or pharmaceutical compositions of the present invention) is preferably a human, more preferably a human patient who comprises one or more genes encoding for a polyQ disease-related protein, said protein comprising a polyQ tract with a pathogenic length, and even more preferably a human patient who suffers from a polyQ disease.

Accordingly, the present invention relates to a method of treating a a polyQ disease, wherein the method comprises administering an effective amount of the nucleic acid molecules, conjugate compounds or pharmaceutical compositions of the invention to a patient suffering from said disease.

The invention also provides for the use of the nucleic acid molecule, a conjugate compound or a pharmaceutical composition of the invention for the manufacture of a medicament, in particular a medicament for use in the treatment of a polyQ disease. In preferred embodiments, the medicament is manufactured in a dosage form for parenteral administration.

The invention also provides for the use of the nucleic acid molecule, a conjugate compound, the pharmaceutical composition of the invention for the manufacture of a medicament wherein the medicament is in a dosage form for intravenous administration.

The present invention also relates to an ex vivo method for preventing aggregation of the polyQ disease-related protein comprising contacting cells expressing polyQ disease-related protein with the nucleic acid molecule, a conjugate compound, the pharmaceutical composition of the invention.

The invention will now be illustrated by the following examples which have no limiting character.

### Example 1: RNA Editing in the CAG Repeat Region

### Introduction

In this experiment, we show that we are able to do RNA editing in the CAG repeat region of the ATXN3 mRNA in Hela cells. The mainly expressed transcript isoform of ATXN3 in Hela cells is ATXN3-254 (ENST00000644486.2). The sequence is shown in SEQ ID NO:7. The variable CAG repeat region is located in position 922-945 of SEQ ID NO: 7.

Materials and methods 7500 Hela cells per well were seeded in a 96-well plate. 24 hours after seeding cells were transfected with a mix of 5 pmol antisense oligonucleotide (ASO) and 0.3 μl Lipofectamine RNAiMAX (Thermo Fisher Scientific) in OptiMEM (Thermo Fisher Scientific). Table 1 shows the tested antisense compounds were tested.

TABLE 1
ASOs:

| | CMP | |
|---|---|---|
| SEQ | ID | |
| ID | NO | Oligonucleotide compound |
| 1 | 1_1 | g*c*u*g*c* |
| | | U*G*C*U*G*C*U*G*C*U*G*C*U*G*C*U*G*CUGCUGCUGCUG |
| | | CCG CUGCUGCUG*C* u*g*c*u*g |
| 2 | 2_1 | g*c*u*g*c* |
| | | U*G*C*U*G*C*U*G*C*U*G*C*U*G*C*U*G*CUGCUGCUGCUG |
| | | CCI CUGCUGCUG*C* u*g*c*u*g |
| 3 | 3_1 | g*c*u*g*c* |
| | | U*G*C*U*G*C*U*G*C*U*G*C*U*G*C*U*G*CUGCUGCUG CCI |
| | | CCI CUGCUGCUG*C* u*g*c*u*g |
| 4 | 4_1 | g*c*u*g*c* |
| | | U*G*C*U*G*C*U*G*C*U*G*C*U*G*C*U*G*CUGCUG CCI CUG |
| | | CCI CUGCUGCUG*C* u*g*c*u*g |
| 5 | 5_1 | g*c*u*g*c* U*I*C*U*G*C*U*I*C*U*G*C*U*I*C*U*G*CUGCUG |
| | | CCI CUG CCI CUGCUG*C*U*I*C* U*G*C*U*I* |
| | | C*U*G*C*U*I*C*U* g*c*u*g*c |
| 6 | 6_1 | g*c*u*g*c* U*G*C*U*G*C*U*G*C*U*G*C*U*G*C*U*G* CCI |
| | | CUG CCI CUG CCI CUG CCI* C*U*G*C* U*G*C*U*G* |
| | | C*U*G*C*U*G*C*U* g*c*u*g*c |

A capital letter represents an RNA nucleoside, a lower case letter represents a 2′-O-methyl-RNA nucleoside, an asterisk (*) represents a phosphorothioate internucleoside linkage, and wherein all other linkages are phosphodiester internucleoside linkages. A bold C indicates a C opposite of the target A.

Cells were harvested after 24 hours with RLT buffer and RNA was isolated using the RNeasy 96 kit (Qiagen). RNA was reverse transcribed using the iScript Select cDNA Synthesis Kit (Bio-Rad) with a transcript specific primer. Before the reverse transcription was performed, an oligonucleotide complementary to the CAG ASO and the transcript specific reverse primer were added to the isolated RNA and incubated at 90° C. for 2 min. The resulting cDNA was amplified by Phusion PCR using the Phusion High Fidelity PCR Master Mix with GC Buffer (Thermo Fisher Scientific) with primers that contain a transcript specific part and a 20 nt overhang serving as primer binding sites for a second PCR. Subsequently the PCR product was diluted 1:10000 in water and a second Phusion PCR was performed using primers with individual barcodes and Illumina adapters for NGS sequencing. After the second PCR, the PCR products of the individual wells were pooled and purified with the Monarch PCR&DNA Cleanup KIT (NEB). The indexed NGS library was sequenced on an illumina mini Seq system according to manufactures instructions.

The generated fastq files were analyzed using the CLC Genomics Workbench Version 20.0.4 software (Qiagen). Initially the part of the reads originated from the primers were trimmed away including the adjacent gg (position 948-949 of SEQ ID NO: 7) leaving behind the ag(cag)ₙgg region.

Initially, the length of the CAG repeat region present in untreated Hela cells was determined. Hela cells contain alleles with 21 and 22 CAG repeats. Furthermore, we noticed the presence of a SNP (“G/C” at position 946 of SEQ ID 7, chr 14 92,071,010) present in a fraction of the mRNAs with 21 CAG repeats. Hela cells are known to contain more than 2 copies of each chromosome, and that is why we can see a SNP in a fraction of the reads.

We defined two new reference sequences containing 21 or 22 CAG repeats, starting at the A of the first CAG and containing the two adjacent G and the 3′ end:

SEQ ID NO: 8: ATXN3 region from ATXN3 mRNA containing 21 CAG repeats:
AGCAGCAGCAGCAGCAGCAGCAGCAGCAGCAGCAGCAGCAGCAGCAGCAGCAGCA
GCAGCAGGG
SEQ ID NO: 9: ATXN3 region from ATXN3 mRNA containing ATXN3 region with 22 CAG
repeats:
AGCAGCAGCAGCAGCAGCAGCAGCAGCAGCAGCAGCAGCAGCAGCAGCAGCAGCA
GCAGCAGCAGGG

The above sequences were used for mapping the reads of the edited mRNAs. The first A in the CAG repeat now called target site position 1 (pos 1) and so forth. So the editable “As” are at position 1, 4, 7, 10 . . . 61.

Following the trimming of the reads leaving behind the ag(cag)ₙgg region, the reads were either 64 or 67 basepairs (bp) long due to the difference in the number of CAG repeats. The reads were separated according their length and were followingly mapped to the appropriate reference sequence (64 bp to SEQ ID 8, 67 bp to SEQ ID 9). The mapping was performed using a low penalty score for mismatches and a minimum fractional sequence identity of 0.65 to allow the mapping of reads with multiple edited As. At least 422 reads were mapped within each sample.

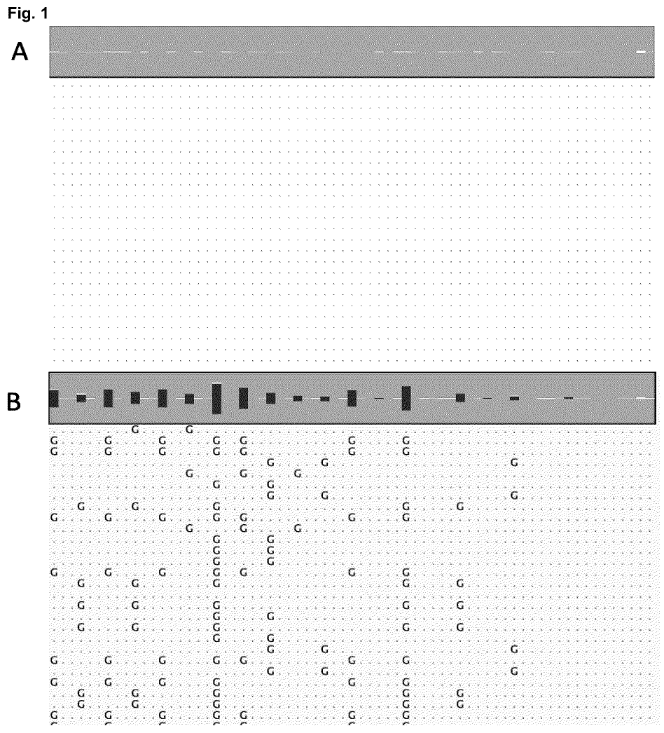

The mapped reads showed extensive editing of As in the ASO treated cells. Both the position and the number of edited As varied within the treatment. FIG. 1 illustrates a representative fraction of the reads in a PBS sample (FIG. 1A) and a sample treated with ASO CMP ID: 4 (FIG. 1B). FIG. 1B shows that an A->G editing has occurred multiple times in the ASO (CMP ID: 4) treated cells.

Further, a variant calling was performed on the mapped reads from each sample. A cut-off of minimum 1% was used as filter in the variant detection. Table 2 shows the percentile of edited “As” following treatment with each antisense compound (see Table 1) and PBS control. The final row shows the percentile of completely unedited mRNAs.

TABLE 3
Proportion of RNA editing at different positions following
ASO treatment. % A to G editing measured at different
position. N/A means no variant call were made, meaning
less than 1% of the reads mismatched at that position.
Bottom row show percentile of reads without any editing.

| | CMP | CMP | CMP | CMP | CMP | CMP | |
|---|---|---|---|---|---|---|---|
| Target | ID | ID | ID | ID | ID | ID | |
| position | 1_1 | 2_1 | 3_1 | 4_1 | 5_1 | 6_1 | PBS |
| 1 | 9.3 | 40.7 | 27.2 | 23.8 | 14.6 | 27.4 | N/A |
| 4 | 69.3 | 8.2 | 48.0 | 15.4 | 18.0 | 23.5 | N/A |
| 7 | N/A | 21.1 | 34.0 | 43.3 | 34.3 | 36.3 | N/A |
| 10 | 60.2 | N/A | 8.0 | 15.9 | 37.4 | 21.5 | N/A |
| 13 | N/A | 7.2 | 19.2 | 40.7 | 34.8 | 36.3 | N/A |
| 16 | 24.2 | 59.8 | 14.8 | 30.9 | 25.1 | 31.0 | N/A |
| 19 | 60.1 | 9.1 | 40.2 | 40.7 | 29.1 | 26.3 | N/A |
| 22 | 2.4 | 8.9 | 3.9 | 36.8 | 32.1 | 25.8 | N/A |
| 25 | 4.9 | 13.5 | N/A | 14.9 | 27.6 | 42.4 | N/A |
| 28 | 60.1 | 8.0 | 10.2 | 16.9 | 41.2 | 22.3 | N/A |
| 31 | 2.2 | 1.8 | 3.9 | 11.4 | 20.4 | 36.9 | N/A |
| 34 | 59.9 | N/A | 16.0 | 34.4 | 36.8 | 33.4 | N/A |
| 37 | 9.1 | N/A | 27.4 | 22.9 | 8.4 | 31.1 | N/A |
| 40 | 8.2 | 46.6 | 4.7 | 31.0 | 29.5 | 38.2 | N/A |
| 43 | 59.9 | N/A | 28.8 | 22.7 | 19.7 | 24.5 | N/A |
| 46 | N/A | N/A | N/A | 12.0 | 6.0 | 11.8 | N/A |
| 49 | N/A | N/A | N/A | N/A | 8.4 | 18.0 | N/A |
| 52 | N/A | N/A | N/A | 8.8 | N/A | 17.1 | N/A |
| 55 | N/A | N/A | N/A | N/A | 15.6 | 3.9 | N/A |
| 58 | N/A | N/A | N/A | 2.1 | 12.1 | 6.5 | N/A |
| 61 | N/A | 17.6 | N/A | N/A | 21.4 | N/A | N/A |
| % | 1.1 | 1.8 | 1.3 | 1.7 | 0.5 | 0.8 | 100.0 |
| Unedited | | | | | | | |

1. A method for treating and/or preventing a polyglutamine (polyQ) disease in a subject in need thereof, comprising administering to the subject a therapeutically effective amount of an antisense oligonucleotide, wherein antisense oligonucleotide is capable of specifically binding to a CAG repeat region of an mRNA encoding a polyQ disease-related protein such that a double stranded RNA is formed which is capable of attracting an Adenosine Deaminase Acting on RNA (ADAR) inserting an A to I exchange into at least one CAG trinucleotide of the CAG repeat region of said mRNA.

2. The method of claim 1, wherein said antisense oligonucleotide is a single-stranded antisense oligonucleotide.

3. The method of claim 1, wherein said antisense oligonucleotide has a length of 20 to 150 nucleotides.

4. The method of claim 1, wherein the antisense oligonucleotide is an RNA, wherein said antisense oligonucleotide comprises a contiguous nucleotide sequence which is at least 90% complementary to a CAG repeat region of an mRNA encoding a polyQ disease-related protein.

5. The method of claim 1, wherein the antisense oligonucleotide comprises a contiguous nucleotide sequence which is at least 90% complementary to an RNA sequence as shown in SEQ ID NO: 10, 11, or 12

6. The method of claim 2, wherein said oligonucleotide, or contiguous nucleotide sequence, comprises at least one mismatch to at least one adenosine in the CAG repeat region.

7. The method of claim 1, wherein said antisense oligonucleotide comprise at least one 2′ sugar modified nucleoside.

8. The method of claim 7, wherein said at least one 2′ sugar modified nucleoside is selected from the group consisting of: 2′-O-alkyl-RNA, 2′-O-methyl-RNA, 2′-O-alkoxy-RNA, 2′-O-methoxyethyl-RNA, 2′-fluoro-RNA, arabino nucleic acid (ANA), 2′-fluoro-ANA, and an LNA nucleoside, and/or wherein said antisense oligonucleotide comprises at least one phosphorothioate internucleoside linkage.

9. The method of claim 1, wherein said antisense oligonucleotide comprises or consists of a nucleic acid sequence as shown in any one of SEQ ID NOs: 1 to 6.

10. The method of claim 9, wherein the oligonucleotide comprises or consists of a contiguous nucleotide sequence, selected from the group consisting of:
(SEQ ID NO: 1)
g*c*u*g*c*U*G*C*U*G*C*U*G*C*U*G*C*U*G*C*U*G* CUGCUGCUGCUG
CCG CUGCUGCUG*C*u*g*c*u*g,
(SEQ ID NO: 2)
g*c*u*g*c* U*G*C*U*G*C*U*G*C*U*G*C*U*G*C*U*G* CUGCUGCUGCUG
CCI CUGCUGCUG*C* u*g*c*u*g,
(SEQ ID NO: 3)
g*c*u*g*c* U*G*C*U*G*C*U*G*C*U*G*C*U*G*C*U*G*CUGCUGCUG CCI
CCI CUGCUGCUG*C* u*g*c*u*g,
(SEQ ID NO: 4)
g*c*u*g*c* U*G*C*U*G*C*U*G*C*U*G*C*U*G*C*U*G*CUGCUG CCI CUG
CCI CUGCUGCUG*C* u*g*c*u*g,
(SEQ ID NO: 5)
g*c*u*g*c* U*I*C*U*G*C*U*I*C*U*G*C*U*I*C*U*G*CUGCUG CCI CUG CCI
CUGCUG*C*U*I*C* U*G*C*U*I* C*U*G*C*U*I*C*U* g*c*u*g*c,
or
(SEQ ID NO: 6)
g*c*u*g*c* U*G*C*U*G*C*U*G*C*U*G*C*U*G*C*U*G* CCI CUG CCI CUG
CCI CUG CCI* C*U*G*C* U*G*C*U*G* C*U*G*C*U*G*C*U* g*c*u*g*c
wherein a capital letter represents a RNA nucleoside, a lower case letter represents a 2′-O-alkyl-RNA nucleoside, and wherein an asterisk (*) represents a phosphorothioate internucleoside linkage, and wherein all other linkages are phosphodiester internucleoside linkages.

11. The method of claim 1, wherein said polyQ disease is selected from the group consisting of: Huntington's disease (HD), Spinocerebellar ataxia (SCA) 1, 2, 3, 6, 7, or 17, Dentatorubralpallidoluysian atrophy, and Kennedy's disease.

12. The antisense oligonucleotide of claim 1, wherein said polyQ disease is
HD and the polyQ disease-related protein is HTT,
SCA 1 and the polyQ disease-related protein is ATXN1,
SCA 2 and the polyQ disease-related protein is ATXN2,
SCA 3 and the polyQ disease-related protein is ATXN3,
SCA 6 and the polyQ disease-related protein is CACNA1A,
SCA 7 and the polyQ disease-related protein is ATXN7,
SCA 12 and the polyQ disease-related protein is PPP2R2B,
SCA 17 and the polyQ disease-related protein is TBP,
Dentatorubralpallidoluysian atrophy and the polyQ disease-related protein is ATN1 or DRPLA; or
Kennedy's disease and the polyQ disease-related protein is AR.

13. The method of claim 12, wherein
the polyQ disease-related protein is HTT, and wherein the mRNA encoding said protein comprises 36 to 250 consecutive CAG repeats,
the polyQ disease-related protein is ATXN1, and wherein the mRNA encoding said protein comprises 49 to 88 consecutive CAG repeats,
the polyQ disease-related protein is ATXN2, and wherein the mRNA encoding said protein comprises 32 to 100 consecutive CAG repeats,
the polyQ disease-related protein is ATXN3, and wherein the mRNA encoding said protein comprises 55 to 86 consecutive CAG repeats,
the polyQ disease-related protein is CACNA1A, and wherein the mRNA encoding said protein comprises 49 to 88 consecutive CAG repeats,
the polyQ disease-related protein is ATXN7, and wherein the mRNA encoding said protein comprises 37 to 306 consecutive CAG repeats,
the polyQ disease-related protein is PPP2R2B, and wherein the mRNA encoding said protein comprises 55 to 78 consecutive CAG repeats,
the polyQ disease-related protein is TBP, and wherein the mRNA encoding said protein comprises 47 to 63 consecutive CAG repeats, and wherein the mRNA encoding said protein comprises 49 to 88 consecutive CAG repeats,
the polyQ disease-related protein is ATN1 or DRPLA; and wherein the mRNA encoding said protein comprises 49 to 88 consecutive CAG repeats, or
the polyQ disease-related protein is AR, and wherein the mRNA encoding said protein comprises 38 to 62 consecutive CAG repeats.

14. The method of claim 1, wherein aggregation of the polyQ disease-related protein is prevented by said inserting an A to I exchange into at least one CAG trinucleotide of the CAG repeat region of the mRNA of the polyQ disease-related protein.

15. A single-stranded antisense oligonucleotide capable of specifically binding to a CAG repeat region of an mRNA encoding a polyQ disease-related protein such that a double stranded RNA is formed which is capable of attracting an Adenosine Deaminase Acting on RNA (ADAR) inserting an A to I exchange into at least one CAG trinucleotide of the CAG repeat region of said mRNA.

16. A pharmaceutical composition comprising the antisense oligonucleotide of claim 15 and a pharmaceutically acceptable excipient.

17. (canceled)

18. An ex vivo method for preventing aggregation of the polyQ disease-related protein comprising contacting cells expressing polyQ disease-related protein with the single-stranded antisense oligonucleotide of claim 15.

19. A polyQ disease-associated polypeptide comprising one or more arginine residues in its polyQ tract.


SEQUENCE LISTING (13 sequences)

SEQ ID NO: 1 - RNA, 52 nt, synthetic construct
  misc_feature 1..52: note = Antisense oligonucleotide
  source 1..52: mol_type = other RNA
gctgctgctgctgctgctgctgctgctgctgctgccgctgctgctgctgctg

SEQ ID NO: 2 - RNA, 52 nt, synthetic construct
  modified_base 1..52: mod_base = OTHER; note = Antisense oligonucleotide
  modified_base 37: mod_base = i
  source 1..52: mol_type = other RNA
gctgctgctgctgctgctgctgctgctgctgctgccnctgctgctgctgctg

SEQ ID NO: 3 - RNA, 52 nt, synthetic construct
  modified_base 1..52: mod_base = OTHER; note = Antisense oligonucleotide
  modified_base 34: mod_base = i
  modified_base 37: mod_base = i
  source 1..52: mol_type = other RNA
gctgctgctgctgctgctgctgctgctgctgccnccnctgctgctgctgctg

SEQ ID NO: 4 - RNA, 52 nt, synthetic construct
  modified_base 1..52: mod_base = OTHER; note = Antisense oligonucleotide
  modified_base 31: mod_base = i
  modified_base 37: mod_base = i
  source 1..52: mol_type = other RNA
gctgctgctgctgctgctgctgctgctgccnctgccnctgctgctgctgctg

SEQ ID NO: 5 - RNA, 65 nt, synthetic construct
  modified_base 1..65: mod_base = OTHER; note = Antisense oligonucleotide
  modified_base 7: mod_base = i
  modified_base 13: mod_base = i
  modified_base 19: mod_base = i
  modified_base 31: mod_base = i
  modified_base 37: mod_base = i
  modified_base 46: mod_base = i
  modified_base 52: mod_base = i
  modified_base 58: mod_base = i
  source 1..65: mol_type = other RNA
gctgctnctgctnctgctnctgctgctgccnctgccnctgctgctnctgctnctgctnctgctgc

SEQ ID NO: 6 - RNA, 65 nt, synthetic construct
  modified_base 1..65: mod_base = OTHER; note = Antisense oligonucleotide
  modified_base 25: mod_base = i
  modified_base 31: mod_base = i
  modified_base 37: mod_base = i
  modified_base 43: mod_base = i
  source 1..65: mol_type = other RNA
gctgctgctgctgctgctgctgccnctgccnctgccnctgccnctgctgctgctgctgctgctgc

SEQ ID NO: 7 - DNA, 6884 bp, Homo sapiens
  source 1..6884: mol_type = genomic DNA
gtgggggccgttggctccagacaaataaacatggagtccatcttccacgagaaacaagaaggctcactttgtgctcaacattgcctgaataacttattgcaaggagaatattttagccctgtggaattatcctcaattgcacatcagctggatgaggaggagaggatgagaatggcagaaggaggagttactagtgaagattatcgcacgtttttacagcagccttctggaaatatggatgacagtggttttttctctattcaggttataagcaatgccttgaaagtttggggtttagaactaatcctgttcaacagtccagagtatcagaggctcaggatcgatcctataaatgaaagatcatttatatgcaattataaggaacactggtttacagttagaaaattaggaaaacagtggtttaacttgaattctctcttgacgggtccagaattaatatcagatacatatcttgcacttttcttggctcaattacaacaggaaggttattctatatttgtcgttaagggtgatctgccagattgcgaagctgaccaactcctgcagatgattagggtccaacagatgcatcgaccaaaacttattggagaagaattagcacaactaaaagagcaaagagtccataaaacagacctggaacgagtgttagaagcaaatgatggctcaggaatgttagacgaagatgaggaggatttgcagagggctctggcactaagtcgccaagaaattgacatggaagatgaggaagcagatctccgcagggctattcagctaagtatgcaaggtagttccagaaacatatctcaagatatgacacagacatcaggtacaaatcttacttcagaagagcttcggaagagacgagaagcctactttgaaaaacagcagcaaaagcagcaacagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataatacctttaaaaaataatttagatattcatactttccaacattatcctgtgtgattacagcatagggtccactttggtaatgtgtcaaagagatgaggaaataagacttttagcggtttgcaaacaaaatgatgggaaagtggaacaatgcgtcggttgtaggactaaataatgatcttccaaatattagccaaagaggcattcagcaattaaagacatttaaaatagttttctaaatgtttctttttcttttttgagtgtgcaatatgtaacatgtctaaagttagggcatttttcttggatctttttgcagactagctaattagctctcgcctcaggctttttccatatagtttgttttctttttctgtcttgtaggtaagttggctcacatcatgtaatagtggctttcatttcttattaaccaaattaacctttcaggaaagtatctctactttcctgatgttgataatagtaatggttctagaaggatgaacagttctcccttcaactgtataccgtgtgctccagtgttttcttgtgttgttttctctgatcacaacttttctgctacctggttttcattattttcccacaattcttttgaaagatggtaatcttttctgaggtttagcgttttaagccctacgatgggatcattatttcatgactggtgcgttcctaaactctgaaatcagccttgcacaagtacttgagaataaatgagcattttttaaaatgtgtgagcatgtgctttcccagatgctttatgaatgtcttttcacttatatcaaaaccttacagctttgttgcaaccccttcttcctgcgccttattttttcctttcttctccaattgagaaaactaggagaagcatagtatgcaggcaagtctccttctgttagaagactaaacatacgtacccaccatgaatgtatgatacatgaaatttggccttcaattttaatagcagttttattttattttttctcctatgactggagctttgtgttctctttacagttgagtcatggaatgtaggtgtctgcttcacatcttttagtaggtatagcttgtcaaagatggtgatctggaacatgaaaataatttactaatgaaaatatgtttaaatttatactgtgatttgacacttgcatcatgtttagatagcttaagaacaatggaagtcacagtacttagtggatctataaataagaaagtccatagttttgataaatattctctttaattgagatgtacagagagtttcttgctgggtcaataggatagtatcattttggtgaaaaccatgtctctgaaattgatgttttagtttcagtgttccctatccctcattctccatctccttttgaagctcttttgaatgttgaattgttcataagctaaaatccaagaaatttcagctgacaacttcgaaaattataatatggtatattgccctcctggtgtgtggctgcacacattttatcagggaaagttttttgatctaggatttattgctaactaactgaaaagagaagaaaaaatatcttttatttatgattataaaatagctttttcttcgatataacagattttttaagtcattattttgtgccaatcagttttctgaagtttcccttacacaaaaggatagctttattttaaaatctaaagtttcttttaatagttaaaaatgtttcagaagaattataaaactttaaaactgcaagggatgttggagtttagtactactccctcaagatttaaaaagctaaatattttaagactgaacatttatgttaattattaccagtgtgtttgtcatattttccatggatatttgttcattacctttttccattgaaaagttacattaaacttttcatacacttgaattgatgagctacctaatataaaaatgagaaaaccaatatgcattttaaagttttaactttagagtttataaagttcatatataccctagttaaagcacttaagaaaatatggcatgtttgacttttagttcctagagagtttttgtttttgtttttgtttttttttgagacggagtcttgctatgtctcccaggctggagggcagtggcatgatctcggctcactacaacttccacctcccgggttcaagcaattctcctgcctcagcctccagagtagctgggattacaggcgcccaccaccacacccggcagatttttgtatttttggtagagacgcggtttcatcatgtttggccaggctggtctcgaactcctgacctcaggtgatccgcctgccttggcctcccaaagtgttgggattacaggcatgagccactgcgcctggccagctagagagtttttaaagcagagctgagcacacactggatgcgtttgaatgtgtttgtgtagtttgttgtgaaattgttacatttagcaggcagatccagaagcactagtgaactgtcatcttggtggggttggcttaaatttaattgactgtttagattccatttcttaattgattggccagtatgaaaagatgccagtgcaagtaaccatagtatcaaaaaagttaaaaattattcaaagctatagtttatacatcaggtactgccatttactgtaaaccacctgcaagaaagtcaggaacaactaaattcacaagaactgtcctgctaagaagtgtattaaagatttccattttgttttactaattgggaacatcttaatgtttaatatttaaactattggtatcatttttctaatgtataatttgtattactgggatcaagtatgtacagtggtgatgctagtagaagtttaagccttggaaataccactttcatattttcagatgtcatggatttaatgagtaatttatgtttttaaaattcagaatagttaatctctgatctaaaaccatcaatctatgttttttacggtaatcatgtaaatatttcagtaatataaactgtttgaaaaggctgctgcaggtaaactctatactaggatcttggccaaataatttacaattcacagaatattttatttaaggtggtgctttttttttttgtccttaaaacttgatttttcttaactttattcatgatgccaaagtaaatgaggaaaaaaactcaaaaccagttgagtatcattgcagacaaaactaccagtagtccatattgtttaatattaagttgaataaaataaattttatttcagtcagagcctaaatcacattttgattgtctgaatttttgatactatttttaaaatcatgctagtggcggctgggcgtggtagctcacgcctgtaatcccagcattttgggaggccgaagtgggtggatcacgaggtcgggagttcgagaccagcttggccaaaatggtgaaaccccatctgtactaaaaactacaaaaattagctgggcgcggtggcaggtgcctgtaatcccagctacctgggagtctgaggcaggagaattgcttgaaccctggcgacagaggatgcagtgagccaagatggtgccactgtactccagactgggcgacagagtgagactctgtctcaaaaaaaaaaaaaaaatcatgctagtgccaagagctactaaattcttaaaaccggcccattggacctgtacagataaaaaatagattcagtgcataatcaaaatatgataattttaaaatcttaagtagaaaaataaatcttgatgttttaaattcttacgaggattcaatagttaatattgatgatctcccggctgggtgcagtggctcacgcctgtaatcccagcagttctggaggctgaggtgggcgaatcacttcaggccaggagttcaagaccagtctgggcaacatggtgaaacctcgtttctactaaaaatacaaaaattagccgggcgtggttgcacacacttgtaatcccagctactcaggaggctaagaatcgcatgagcctaggaggcagaggttgcagagtgccaagggctcaccactgcattccagcctgcccaacagagtgagacactgtttctgaaaaaaaaaaatatatatatatatatatatatgtgtgtatatatatatgtatatatatatgacttcctattaaaaactttatcccagtcgggggcagtggctcacgcctgtaatcccaacactttgggaggctgaggcaggtggatcacctgaagtccggagtttgagaccagcctggccaacatggtgaaaccccatctctactaaaaatacaaaacttaagccaggtatggtggcgggcacctgtaatcccagttacttgggaggctgaggcaggagaatcgtttaaacccaggaggtggaggttgcagtgagctgagatcgtgccattgcactctagcctgggcaacaagagtaaaactccatcttaaaggtttgtttgtttttttttaatccggaaacgaagaggcgttgggccgctattttctttttctttctttctttctttctttttttttttttctgagacggagtctagctctgctgcccaggctggagtacaatgacacgatgttggctcactgcaacctccacctcctgggttcaagcgattctcctgcctcagcctcccaagtacctgggattacaggcacctgccactacacctggcgaatatttgttttttttagtagagacgggcttttaccatgttaggctggtctcaaactcctgacctcaggtgatctgcctgccttggcctcccaaagtgctgggattacaggtgcaggccaccacacccggccttgggccactgttttcaaagtgaattgtttgttgtatcgagtccttaagtatggatatatatgtgaccctaattaagaactaccagattggatcaactaatcatgtcagcaatgtaaataactttatttttcatattcaaaataaaaactttcttttatttctggcccctttataaccagcatctttttgctttaaaaaatgacctggctttgtatttttttagtcttaaacataataaaaatatttttgttctaatttgctttcatgagtgaagattattgacatcgttggtaaattctagaattttgattttgttttttaatttgaagaaaatctttgctattattattttttccaagtggtctggcattttaagaattagtgctaataacgtaacttctaaatttgtcgtaattggcatgtttaatagcatatcaaaaaacattttaagcctgtggattcatagacaaagcaatgagaaacattagtaaaatataaatggatattcctgatgcatttaggaagctctcaattgtctcttgcatagttcaaggaatgttttctgaatttttttaatgctttttttttttttgaaagaggaaaacatacatttttaaatgtgattatctaatttttacaacactgggctattaggaataactttttaaaaattactgttctgtataaatatttgaaattcaagtacagaaaatatctgaaacaaaaagcattgttgtttggccatgatacaagtgcactgtggcagtgccgcttgctcaggacccagccctgcagcccttctgtgtgtgctccctcgttaagttcatttgctgttattacacacacaggccttcctgtctggtcgttagaaaagccgggcttccaaagcactgttgaacacaggattctgttgttagtgtggatgttcaatgagttgtattttaaatatcaaagattattaaataaagataatgtttgcttttcta

SEQ ID NO: 8 - DNA, 64 bp, Homo sapiens
  source 1..64: mol_type = genomic DNA
agcagcagcagcagcagcagcagcagcagcagcagcagcagcagcagcagcagcagcagcaggg

SEQ ID NO: 9 - DNA, 67 bp, Homo sapiens
  source 1..67: mol_type = genomic DNA
agcagcagcagcagcagcagcagcagcagcagcagcagcagcagcagcagcagcagcagcagcaggg

SEQ ID NO: 10 - RNA, 39 nt, synthetic construct
  misc_feature 1..39: note = Target sequence
  source 1..39: mol_type = other RNA
cagcagcagcagcagcagcagcagcagcagcagcagcag

SEQ ID NO: 11 - RNA, 57 nt, synthetic construct
  misc_feature 1..57: note = Target sequence
  source 1..57: mol_type = other RNA
cagcagcagcagcagcagcagcagcagcagcagcagcagcagcagcagcagcagcag

SEQ ID NO: 12 - RNA, 69 nt, synthetic construct
  misc_feature 1..69: note = Target sequence
  source 1..69: mol_type = other RNA
cagcagcagcagcagcagcagcagcagcagcagcagcagcagcagcagcagcagcagcagcagcagcag

SEQ ID NO: 13 - RNA, 30 nt, synthetic construct
  variation 1..30: note = Antisense oliginonucleotide
  variation 2: note = n is c or u
  variation 3: note = n is g or i
  variation 5: note = n is c or u
  variation 6: note = n is g or i
  variation 8: note = n is c or u
  variation 9: note = n is g or i
  variation 11: note = n is c or u
  variation 12: note = n is g or i
  variation 14: note = n is c or u
  variation 15: note = n is g or i
  variation 17: note = n is c or u
  variation 18: note = n is g or i
  variation 20: note = n is c or u
  variation 21: note = n is g or i
  variation 23: note = n is c or u
  variation 24: note = n is g or i
  variation 26: note = n is c or u
  variation 27: note = n is g or i
  variation 29: note = n is c or u
  variation 30: note = n is g or i
  source 1..30: mol_type = other RNA
cnncnncnncnncnncnncnncnncnncnn